(19) United States
(12) Patent Application Publication
(10) Pub. No.: US 2022/0399124 A1
(43) Pub. Date: Dec. 15, 2022

(54) RISK DETERMINATION FOR A CT-EXAMINATION

(71) Applicant: Siemens Healthcare GmbH, Erlangen (DE)

(72) Inventors: Yufan ZHAO, Erlangen (DE); Yu Wen KUO, Shanghai (CN); Ralf GUTJAHR, Nuernberg (DE)

(73) Assignee: Siemens Healthcare GmbH, Erlangen (DE)

(21) Appl. No.: 17/835,005

(22) Filed: Jun. 8, 2022

(30) Foreign Application Priority Data
Jun. 11, 2021 (EP) 21179099.3

(51) Int. Cl. G16H 50/30; G16H 10/60

(52) U.S. Cl. CPC G16H 50/30; G16H 10/60

(57) ABSTRACT

Embodiments of the present invention describes a risk indicator device for a CT-examination comprising a data interface configured to receive a dataset of a patient, the dataset comprising patient demographic data, data about previous treatments and data about medication of the patient; a risk indicator unit configured to evaluate a risk-probability for the CT-examination based on the patient demographic data, the data about the previous treatments and the data about the medication of the patient; and an output unit configured to output at least one of output information or control data based on the evaluated risk-probability. Embodiments of the present invention further describe a respective method, a related risk prediction model, a patient scheduler and a CT-system.

### CROSS-REFERENCE TO RELATED APPLICATION(S)

The present application hereby claims priority under 35 U.S.C. § 119 to European patent application number EP 21179099.3 filed Jun. 11, 2021, the entire contents of which are hereby incorporated herein by reference.

### FIELD

Embodiments of the present invention relate to a risk indicator device and a method for risk determination for a CT-examination, especially for risk indication of a CT-examination. Thus, embodiments of the present invention is advantageous for predicting (and displaying) risks for a patient going through a CT-examination.

### BACKGROUND

Computed tomography (CT) is a medical imaging technique used in radiology to get detailed 2D or 3D images of the body for diagnostic purposes. CT-examinations are an important backbone of modern medicine.

CT scanners use a rotating X-ray tube and a number of detectors placed opposite of the tube to measure attenuations of the X-rays when passing a patient or an object inside the scanner. Typically, there are made multiple X-ray measurements taken from different angles in order to reconstruct 3D images of a region of interest.

Although CT-examinations are relatively safe procedures, there are nevertheless some risks, since there is used ionizing radiation (X-rays) for a CT-scan and sometimes contrast agents are applied to a patient to visualize certain regions. These risks are different for different patients. For example, children are more sensitive to radiation exposure than adults another example is a person who are allergic to contrast agents resulting in a (sometimes life-threatening) allergic reaction during or after examination.

At a typical contrast enhanced examination (“Enhanced CT”), a contrast agent is injected into a vein, a CT scan is performed, and the resulting data is processed through a computer to obtain an image. During this examination, the following main risks may occur. First, the contrast agent can cause allergies to the human body and may cause transient damage to the kidney. Second, CT scans are radiographic scans, which can also cause certain damage to the body. In addition, when the contrast agent is injected, a large-dose bolus is required for a short time. Some patients with heart insufficiency may experience aggravated heart failure.

Concerning these risks, patients may be informed previous the examination. However, analysis shows that often patients are not given information about the risks, benefits, and radiation dose for a CT scan (see e.g. Lee, C.I. et al. “Diagnostic CT Scans: Assessment of Patient, Physician, and Radiologist Awareness of Radiation Dose and Possible Risks”, RSNA, Radiology, Vol. 231, No. 2, 2004). It seems that patients, physicians, and radiologists alike are unable to provide accurate estimates of CT risks regardless of their experience level.

Currently, patients have to tell a doctor about any sensitivities to medications or any kidney problems and other factors that may increase the patient's risk. There are many factors that are taken into consideration. But mainly it depends on the doctor's subjective judgement to rate the risk of a patient of a CT scan. There are various questionnaires in different format and many times in paper format to document the evaluation. But until now there is no systematic and automatic way of evaluating patient's risk and indicate the risks directly on a CT scan workflow.

### SUMMARY

Current patient care functionalities focus on automatic dose modulation and contrast medium administration during the scan. But there are no known functions that achieve to predict patient risk with automatic integration of data sources and machine learning models.

Embodiments of the present invention improve the known devices and methods to facilitate a better risk determination for a CT-examination.

This is achieved by a risk indicator device according to embodiments of the present invention, a method according to embodiments of the present invention, a risk prediction model according embodiments of the present invention, a patient scheduler according to embodiments of the present invention and a CT-system according to embodiments of the present invention.

According to at least one example embodiment, a risk indicator device for a CT-examination comprises a data interface configured to receive a dataset of a patient, the dataset comprising patient demographic data, data about previous treatments and data about medication of the patient; a risk indicator unit configured to evaluate a risk-probability for the CT-examination based on the patient demographic data, the data about the previous treatments and the data about the medication of the patient; and an output unit configured to output at least one of output information or control data based on the evaluated risk-probability.

According to at least one example embodiment, the risk indicator unit comprises a risk prediction model trained for risk prediction.

According to at least one example embodiment, the risk indicator unit is configured to evaluate the risk-probability of at least one of an overdose or of contrast allergic reactions of a patient in the course of a following CT-examination, and the risk indicator unit is configured to evaluate the risk-probability of contrast allergic reactions by using additional information of the dataset concerning at least one of (1) allergic dispositions, (2) recent lab results or (3) previous CT-examination and diagnosis of the patient.

According to at least one example embodiment, the data interface is configured to receive medical data types of different structures for accessing data from different sources, wherein the medical data types including data from electronic medical records, cardiovascular information systems, radiology information systems or laboratory information systems.

According to at least one example embodiment, the output unit is configured to output control data for a computed tomography (CT)-system, wherein the control data includes at least one of automatic adjust contrast injection parameters or scan parameters, the control data being based on the evaluated risk-probability.

According to at least one example embodiment, the risk indicator unit is configured to evaluate risk-probability by using multivariate analysis.

At least one example embodiment provides a method for risk determination for a computed tomography (CT)-examination of a patient, comprising receiving a dataset of a patient, the dataset comprising patient demographic data, data about previous treatments and data about medication of the patient; evaluating a risk-probability for the CT-examination of the patient, based on the patient demographic data, the data about the previous treatments and the data about the medication of the patient; and outputting at least one of output information or control data based on the evaluated risk-probability.

According to at least one example embodiment, the risk probability is based on the patient demographic data, the patient demographic data including at least one of gender, age, weight, race, or pregnancy, the data about previous treatments, the data about previous treatments identifying at least one of central venous catheter, kidney surgery, chemotherapy or dialysis, and the data about the medication, the data about the medication identifying Glucophage or anti-inflammatory drugs, the patient demographic data, the data about previous treatments and the data about the medication concerning the respective patient being examined by the CT-examination, wherein the method is configured to determine a risk of overdose.

According to at least one example embodiment, the evaluating includes determining a risk-probability of contrast allergic reactions of a patient, wherein the risk-probability of contrast allergic reactions being based on at least one of allergic dispositions of the respective patient, recent lab results of the respective patient, or previous CT-examination and diagnosis of the respective patient.

According to at least one example embodiment, the evaluating the risk-probability is performed by a risk indicator device, the risk indicator device including a risk prediction model that is trained online by providing a dataset of at least one examination in a CT-system and data about incidents during the CT-examination.

According to at least one example embodiment, the risk prediction model is a machine-learning model trained on a multiplicity of training datasets, each training dataset comprising at least patient demographic data, data about previous treatments, data about medication of a patient, and at least one of (i) data about allergic dispositions, (ii) recent lab results or (iii) previous CT-examination and diagnosis of this patient, and a ground truth, the ground truth indicating at the respective training dataset the occurrence of an incident, wherein the risk prediction model has the architecture of a decision tree.

According to at least one example embodiment, a patient scheduler comprises the risk indicator device of embodiments of the present invention.

According to at least one example embodiment, a computed tomography (CT)-system comprises the risk indicator device of embodiments of the present invention.

According to at least one example embodiment, a computer program product comprises computer-readable instructions, when executed by a control device for a magnetic resonance imaging system, are configured to cause the magnetic resonance imaging device to perform a method of embodiments of the present invention.

According to at least one example embodiment, a computer-readable medium on which is stored program elements that, when executed by a computer unit, are configured to cause the computer unit to perform a method of embodiments of the present invention.

### BRIEF DESCRIPTION OF THE DRAWINGS

Other objects and features of embodiments of the present invention will become apparent from the following detailed descriptions considered in conjunction with the accompanying drawings. It is to be understood, however, that the drawings are designed solely for the purposes of illustration and not as a definition of the limits of the present invention.

FIG. 1 shows a simplified CT system with a system according to an embodiment of the present invention.

FIG. 2 shows a block diagram of the process flow of a preferred method according to an embodiment of the present invention.

FIG. 3 shows a simplified example for the usage of a dataset.

FIG. 4 shows a usage of different data types.

FIG. 5 shows a possible output of the risk indicator device in a patient scheduler.

In the diagrams, like numbers refer to like objects throughout. Objects in the diagrams are not necessarily drawn to scale.

### DETAILED DESCRIPTION

Embodiments of the present invention relate to a risk indicator device designed for risk determination for a CT-examination, comprising:
a data interface designed for receiving a dataset of a patient, the dataset comprising patient demographic data, data about previous treatments and data about medication of the patient,
a risk indicator unit designed to evaluate a risk-probability for the CT-examination based on the dataset of the patient, in particular based on the patient demographic data, the data about the previous treatments and the data about the medication of the patient,
an output unit designed to output output information and/or control data based on the evaluated risk-probability.

The risk indicator device could be an independent device. However, it could also be present as a function in a higher-ranking device. For example, the risk indicator device could be a function of a patient scheduler or a CT control device. In this case, the risk indicator device could also be designated “risk indicator function”.

The risk indicator device is preferably designed for determining contrast allergic risks and overdose risks. For both determinations, patient demographic data, data about previous treatments and data about medication of the patient are used. For effectively determining contrast allergic risks, it is very advantageous to also have data of the group allergic dispositions (of the patient), recent lab results (of examinations of the patient), previous exams and/or diagnosis (of the patient).

The overdose may be, for example, an overdose of radiation, in particular x-ray radiation, and/or an overdose of contrast agent.

Preferred patient demographic data comprises data concerning gender, age, weight, race, or pregnancy of the patient.

Preferred data of allergic dispositions comprises data concerning the existence of asthma, iodine-allergy, or beta blocker-allergy of the patient.

Preferred recent lab results comprises data concerning lab results of the patient, especially the existence of creatinine in the patient.

Preferred previous exam and diagnosis data comprises data concerning diseases of the patient, especially hypertension, heart failure, myeloma, or diabetes.

Preferred data about previous treatments comprises data concerning interventions (executed or necessary) or the insertion of objects into the patient, especially central venous catheter, kidney surgery, chemotherapy, or dialysis.

Preferred data about medication comprises data concerning medication taken by the patient (and especially still in the patient's body), especially Glucophage or anti-inflammatory drugs or drugs connected with the previously mentioned data (e.g. previous treatments or allergic dispositions).

Suitable data interfaces are known to the skilled person, e.g. RIS (Radiology Information System) or PACS (Picture Archiving and Communication System). In the following the term “dataset” is used for a set comprising patient demographic data, data about previous treatments and data about medication of the patient, wherein this data is used for further determinations. The dataset may also comprise data selected from the group allergic dispositions (of the patient), recent lab results (of examinations of the patient), previous exams and/or diagnosis (of the patient), that may also be used for further determinations.

The risk indicator unit uses the dataset to determine the risk-probability based on the data comprised by the dataset. It preferably determines at least the risk-probability for contrast allergic risks and/or for overdose risks. This could be achieved by a mathematical function weighting scores of the different data types of the dataset (e.g. by a function of the parameters) or by a machine learning model (a “risk prediction model” as described in the following) that had been trained on datasets with respective types of data and labels (as ground truth) whether an incident (especially concerning overdose and/or the occurrence of allergic reaction on a contrast agent).

Suitable output units are known to the skilled person and may e.g. be displays or speakers. The output information may be a risk-score, graphical items indicating a risk (e.g. a red warning sign) or acoustic information e.g. a warning sound. However, the output may also be control data for automatically controlling a CT-system, e.g. reducing the dose if a risk of overdose has been determined or reducing the amount of contrast agent if a risk for an allergic reaction was indicated.

Embodiments of the present invention further relates to a method for risk determination for a CT-examination of a patient, comprising the following steps: - receiving a dataset of a patient, the dataset comprising patient demographic data, data about previous treatments and data about medication of the patient (see above), evaluating a risk-probability for the CT-examination of the patient based on the dataset of the patient, in particular based on the patient demographic data, the data about the previous treatments and the data about the medication of the patient, with a risk indicator unit of a risk indicator device according to embodiments of the present invention,
outputting information and/or control data based on the evaluated risk-probability.

The method for risk determination for a CT-examination of a patient may be, for example, a computer-implemented method.

It should be noted that it is known, what examination should be performed and, thus, information about the planned CT examination, such as radiation dose, intensity, energy and duration of radiation, region of the patient to be examined and contrast agent applied, is well known, also. It is clear that this information is also used for evaluating a risk-probability for a CT-examination of the patient. Thus, for evaluating the risk of an overdose, it is clear that also radiation dose of the planned CT examination, especially as well as radiation energy and other known parameters, is used for evaluation. For evaluating risk of allergic reactions, information about the type and dose of contrast agent that should be applied for the examination is used for evaluation.

With the output (of control data) a CT-system may be controlled by inputting the output information into the control device of the CT-system.

Thus, risk factors can be shown directly to a CT operator and/or can be used to control a CT-system.

A risk prediction model according to embodiments of the present invention is designed for risk-prediction, especially designed for a risk indicator device according to embodiments of the present invention or designed to perform a method according to embodiments of the present invention. The risk prediction model is a machine learning model that is trained on a multiplicity of training datasets. Each training dataset comprising at least:
Patient demographic data, data about previous treatments and data about medication of a patient, and especially also data about allergic dispositions and/or recent lab results and/or previous exam and diagnosis of this patient, and
A ground truth, indicating at the respective training dataset the occurrence of an incident, especially concerning overdose and/or the occurrence of allergic reaction on a contrast agent.

The risk prediction model has preferably the architecture of a decision tree. The risk prediction model could also be a multivariate analysis.

A patient scheduler according to embodiments of the present invention comprises a risk indicator device according to embodiments of the present invention and is preferably designed to perform a method according to embodiments of the present invention. A “patient scheduler” in the sense of embodiments of the present invention is a software module for scheduling patients, i.e. planning CT-exams for a number of patients. The risk indication device is preferably implemented in form of a risk indication function in the patient scheduler, e.g. integrated in the program code with its functionality. It could also be connected to the patient scheduler via a data interface connected to an external risk indication device (e.g. in a cloud).

A control device according to embodiments of the present invention for controlling a CT-system comprises a risk indicator device according to embodiments of the present invention. Alternatively or additionally it is designed to perform the method according to embodiments of the present invention. The control device may comprise additional units or devices for controlling components of a CT-system.

A CT-system (computing tomography system) according to embodiments of the present invention comprises a risk indicator device according to embodiments of the present invention and especially a control device according to embodiments of the present invention.

Furthermore, we believe that not all data types are used for every sort of risk, since e.g. allergic dispositions are probably not used to evaluate radiation-connected risks. Thus, we believe that a device only used for one special risk does not use all of the listed data types.

Some units or modules of the risk indicator device or the control device mentioned above can be completely or partially realized as software modules running on a processor of a computer or a control device. A realization largely in the form of software modules can have the advantage that applications already installed on a computer can be updated, with relatively little effort, to install and run these units of the present application. Embodiments of the present invention also provide a computer program product with a computer program that is directly loadable into the memory of a computer of a medical imaging system, and which comprises program units to perform the steps of the inventive method when the program is executed by the computer. In addition to the computer program, such a computer program product can also comprise further parts such as documentation and/or additional components, also hardware components such as a hardware key (dongle etc.) to facilitate access to the software.

A computer readable medium such as a memory stick, a hard-disk or other transportable or permanently installed carrier can serve to transport and/or to store the executable parts of the computer program product so that these can be read from a processor unit of a control device or a system. A processor unit can comprise one or more microprocessors or their equivalents.

Particularly advantageous embodiments and features of the present invention are given by the dependent claims, as revealed in the following description. Features of different claim categories may be combined as appropriate to give further embodiments not described herein.

According to a preferred risk indicator device, its risk indicator unit comprises a risk prediction model trained for risk prediction (see above). This risk prediction model allows a very accurate and easy risk prediction. However, its training demands some effort.

According to a preferred risk indicator device, the risk indicator unit is designed to evaluate the risk-probability of an overdose and/or of contrast allergic reactions of a patient in the course of a following CT-examination. It is preferred that the risk indicator unit is designed to evaluate the risk-probability of contrast allergic reactions by using additional information of the dataset concerning allergic dispositions and/or recent lab results and/or previous exam and diagnosis of the patient.

A preferred risk indicator device is designed to control a CT-system with the control data. This could be easily achieved by including the risk indicator device in above-described control device or connecting the risk indicator device with such control device. The control data of the output of the risk indicator device should be designed such that they are able to control a CT-system. However, a control device may also act as interpreter for a CT-system for interpreting the control data of the risk indicator device. This has the advantage that the risk-probability is not only indicated but may have a direct influence on the control of a CT-system. Preferably, an examination could only be performed when there is no risk, or the risk-probability lies below a certain threshold. In the case of a risk or a risk-probability above a certain threshold, the system is preferably designed to blocking examination and/or automatically adjusting scan parameters and/or triggering an auditing mechanism or a notification mechanism. This may be achieved by directly controlling the CT-system or by communication with the control device of a CT-system. For example, a CARE-Alert may be outputted what has a direct influence on the control of a CT-system.

A preferred risk indicator device comprises a data interface designed to receive medical data types of different structures, especially a FHIR-interface (FHIR: “Fast Healthcare Interoperability Resources”), for accessing data from different sources. Preferred data types are data from electronic medical records (EMR), Cardiovascular Information Systems (CVIS), Radiology Information Systems (RIS) or Laboratory information systems (LIS). Regardless of the data sources, be it in EMR, RIS or LIS, one can use a standard FHIR interface query and implementation guide to fetch the data to the risk indicator device.

The result could be fed to further systems, e.g. “myExam Companion” and other scan automation mechanisms.

A preferred risk indicator device is designed to output control data for a CT-system. This control data is preferably designed to automatically adjust contrast injection parameters and/or scan parameters depending on the evaluated risk-probability. Thus, dose of radiation or of contrast agent will be adjusted automatically to keep the patient safe from harm automatically.

According to a preferred risk indicator device, the risk indicator unit is designed to evaluate risk-probability by using multivariate analysis.

According to a preferred method, the risk-probability (preferably at least for a risk of overdose) is determined based on
patient demographic data, especially gender, age, weight, race, pregnancy
data about previous treatments, especially central venous catheter, kidney surgery, chemotherapy, dialysis, and
data about medication, especially Glucophage or anti-inflammatory drugs. It should be noted that this concerns the patient that is being examined by the CT-examination.

Preferably, the method is designed to determine the risk of contrast allergic reactions of a patient (alone or along with other risks), wherein the risk-probability is preferably also determined based on
allergic dispositions, especially asthma, iodine, beta blocker and/or
recent lab results, especially creatinine level, and/or
previous exam and diagnosis, especially hypertension, heart failure, myeloma, diabetes, of the respective patient.

For example, a risk-probability of contrast-induced nephropathy is evaluated by using multivariate analysis. This is achieved by looking at the basal Serum creatinine, appearance of a shock, gender (e.g. female), multivessel PCI, and diabetes mellitus as input parameters.

According to a preferred method, the risk indicator device comprises a risk prediction model. This risk prediction model is trained online by providing a dataset of a patient actually examined in a CT-system and data about incidents during the examination. It is especially preferred that a risk-probability for this patient is determined before the examination and compared with possible incidents during examination after the examination. Thus, online-learning is possible.

The method may also include elements of “cloud computing”. In the technical field of “cloud computing”, an IT infrastructure is provided over a data-network, e.g. a storage space or processing power and/or application software. The communication between the user and the “cloud” is achieved by data interfaces and/or data transmission protocols.

In the context of “cloud computing”, in a preferred embodiment of the method according to the present invention, provision of data via a data channel (for example a data-network) to a “cloud” takes place. This “cloud” includes a (remote) computing system, e.g. a computer cluster that typically does not include the user's local machine. This cloud can be made available in particular by the medical facility, which also provides the medical imaging systems. In particular, the image acquisition data is sent to a (remote) computer system (the “cloud”) via a RIS (Radiology Information System) or a PACS (Picture Archiving and Communication System).

Within the scope of a preferred embodiment of the risk indicator device according to the present invention, this device, at least the risk prediction module, are present on the “cloud” side. A preferred system further comprises, a local computing unit connected to the system via a data channel (e.g. a data-network, particularly configured as RIS or PACS). The local computing unit includes at least one data receiving interface to receive data. Moreover, it is preferred if the local computer additionally has a transmission interface in order to send data to the system.

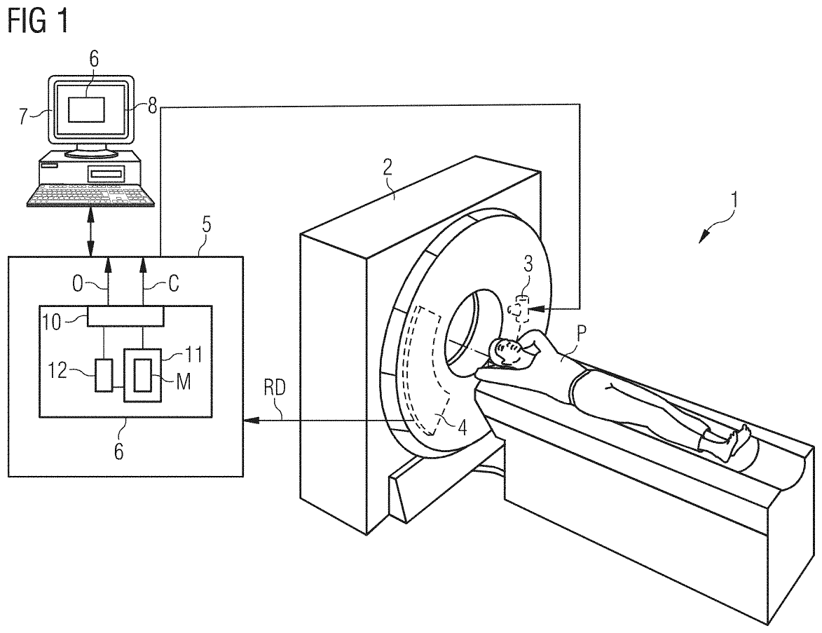

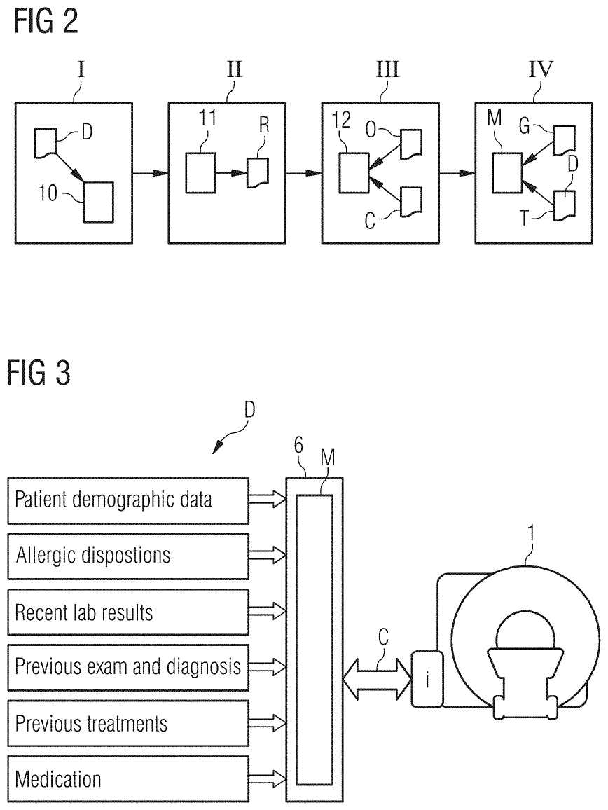

FIG. 1 shows a simplified computer tomography system 1 with a control device 5 comprising a risk indicator device 6 for risk determination for a CT-examination, designed to perform the method according to the invention (see FIG. 2). The computer tomography system 1 has in the usual way a scanner 2 with a gantry, in which an x-ray source 3 with a detector 4 rotates around a patient and records raw data RD that is later reconstructed to images by the control device 5. A patient P lying on a patient bed ready for examination.

In this figure, only those components are shown that are essential for explaining the invention. In principle, such imaging systems and associated control devices are known to the person skilled in the art and therefore do not need to be explained in detail.

A user can interact with the computer tomography system 1 by using terminal 7 that is able to communicate with the control device 5. This terminal 7 can also be used to examine results of the risk indicator device 6 according to the invention or to provide data for the risk indicator device 6.

Although the risk indicator device 6 is shown here in the control device of the CT-system, it could also be present in the terminal 7, e.g. in the patient scheduler 8 as indicated.

The system 6 comprises the following components:

A data interface 10 designed for receiving a dataset D (see FIG. 2) of the patient P to be examined. The data interface 10 shown here is also used for communicating with the CT-system 1 and sending output information 0 and control data C from the output unit 12 of the risk indicator device 6 to the CT-system 1.

Preferably, the data interface 10 is designed to receive medical data types of different structures. It is preferably a FHIR-interface, for accessing data from different sources. Preferred data types are data from electronic medical records, Cardiovascular Information Systems, Radiology Information Systems or Laboratory information systems.

A risk indicator unit 11 designed to evaluate a risk-probability R for the CT-examination. The risk indicator unit 11 here comprises a risk prediction model M trained for risk prediction. Preferably, the risk-probability R of an overdose and/or of contrast allergic reactions of the patient P in the course of the following CT-examination are evaluated.

An output unit 12 that is designed to output output information 0 and/or control data C based on the evaluated risk-probability R. When outputting control data C, the control data C is preferably designed to automatically adjust contrast injection parameters and/or scan parameters, depending on the evaluated risk-probability R.

The components of the system preferably appear to be software modules.

FIG. 2 shows a block diagram of the process flow of a preferred method for risk determination for a CT-examination of a patient.

In step I, a dataset D of a patient P is received by the data interface 10 of the risk indicator device 6 (see FIG. 1). The dataset D comprises patient demographic data, data about previous treatments and data about medication of the patient.

In step II, a risk-probability R for a CT-examination is evaluated based on the patient demographic data, data about previous treatments and data about the medication of the patient. This is achieved with the risk indicator unit 11 of the risk indicator device 6.

In step III output information 0 and control data C are outputted based on the evaluated risk-probability R.

In step IV, a risk prediction model M of the risk indicator device 6 (see FIG. 1) is trained online by providing a dataset D of the patient P examined actually examined in the CT-system 1. This dataset D is here used as training dataset T. In addition, a ground truth G about incidents during the CT-examination is added to train the risk prediction model M.

FIG. 3 shows a simplified example for the usage of a dataset D by a risk prediction model M of a risk indicator device 6. Patient demographic data, data about allergic dispositions, recent lab results and previous CT-examination and diagnosis as well as data about previous treatments and medication are inputted into the risk prediction model M. A risk-probability R is then evaluated by the risk prediction model M, e.g. by a method as shown in FIG. 2, and depending on the evaluated risk-probability R, control data C is sent to a CT-system 1.

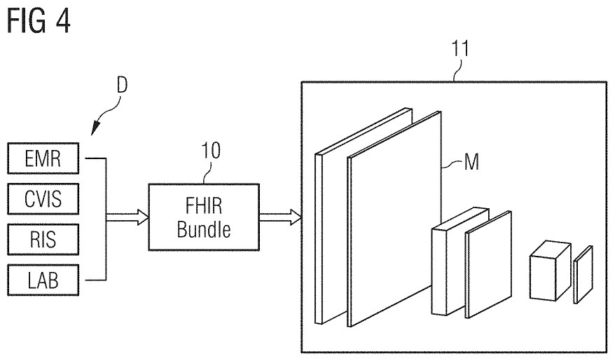

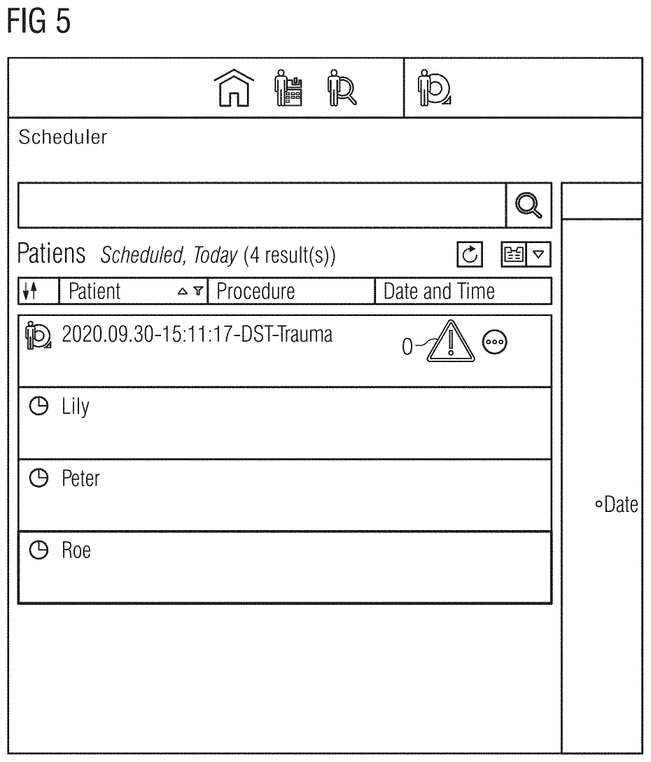

FIG. 4 shows a usage of different data types. The risk indicator device 6 here comprises a special data interface 10 (FHIR-interface here designated as “FHIR bundle”) that is designed to receive medical data types of different structures. The inputted dataset D here comprises data from electronic medical records “EMR”, Cardiovascular Information Systems “CVIS”, radiology information systems “RIS” and lab data from laboratory information systems “LAB”. This data is used by a risk prediction model M in a risk indicator unit 11.

FIG. 5 shows a possible output of the risk indicator device 6 in a patient scheduler 8 (see FIG. 1). Here a possible output of the patient scheduler 8 is shown with a warning sign as output information 0 indicating a certain risk for a patient P for the next CT examination.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

[001.03] In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

1. A risk indicator device for a CT-examination, comprising:
a data interface configured to receive a dataset of a patient, the dataset comprising patient demographic data, data about previous treatments and data about medication of the patient;
a risk indicator unit configured to evaluate a risk-probability for the CT-examination based on the patient demographic data, the data about the previous treatments and the data about the medication of the patient; and
an output unit configured to output at least one of output information or control data based on the evaluated risk-probability.

2. The risk indicator device according to claim 1, wherein the risk indicator unit comprises a risk prediction model trained for risk prediction.

3. The risk indicator device according to claim 1, wherein
the risk indicator unit is configured to evaluate the risk-probability of at least one of an overdose or of contrast allergic reactions of a patient in the course of a following CT-examination, and
the risk indicator unit is configured to evaluate the risk-probability of contrast allergic reactions by using additional information of the dataset concerning at least one of (1) allergic dispositions, (2) recent lab results or (3) previous CT-examination and diagnosis of the patient.

4. The risk indicator device according to claim 1, wherein
the data interface is configured to receive medical data types of different structures for accessing data from different sources, wherein the medical data types including data from electronic medical records, cardiovascular information systems, radiology information systems or laboratory information systems.

5. The risk indicator device according claim 1, wherein the output unit is configured to output control data for a computed tomography (CT)-system, wherein the control data includes at least one of automatic adjust contrast injection parameters or scan parameters, the control data being based on the evaluated risk-probability.

6. The risk indicator device according to claim 1, wherein the risk indicator unit is configured to evaluate risk-probability by using multivariate analysis.

7. A method for risk determination for a computed tomography (CT)-examination of a patient, comprising:
receiving a dataset of a patient, the dataset comprising patient demographic data, data about previous treatments and data about medication of the patient;
evaluating a risk-probability for the CT-examination of the patient, based on the patient demographic data, the data about the previous treatments and the data about the medication of the patient; and
outputting at least one of output information or control data based on the evaluated risk-probability.

8. The method according to claim 7, wherein the risk probability is based on
the patient demographic data, the patient demographic data including at least one of gender, age, weight, race, or pregnancy, the data about previous treatments, the data about previous treatments identifying at least one of central venous catheter, kidney surgery, chemotherapy or dialysis, and the data about the medication, the data about the medication identifying Glucophage or anti-inflammatory drugs,
the patient demographic data, the data about previous treatments and the data about the medication concerning the respective patient being examined by the CT-examination, wherein the method is configured to determine a risk of overdose.

9. The method according to claim 7, wherein the evaluating includes,
determining a risk-probability of contrast allergic reactions of a patient, wherein the risk-probability of contrast allergic reactions being based on at least one of allergic dispositions of the respective patient, recent lab results of the respective patient, or previous CT-examination and diagnosis of the respective patient.

10. The method according to claim 7, wherein the evaluating the risk-probability is performed by a risk indicator device, the risk indicator device including a risk prediction model that is trained online by providing a dataset of at least one examination in a CT-system and data about incidents during the CT-examination.

11. The risk indicator device of claim 2, wherein the risk prediction model is a machine-learning model trained on a multiplicity of training datasets, each training dataset comprising at least patient demographic data, data about previous treatments, data about medication of a patient, and at least one of (i) data about allergic dispositions, (ii) recent lab results or (iii) previous CT-examination and diagnosis of this patient, and
a ground truth, the ground truth indicating at the respective training dataset the occurrence of an incident, wherein the risk prediction model has the architecture of a decision tree.

12. A patient scheduler comprising the risk indicator device according to claim 1.

13. A computed tomography (CT)-system comprising the risk indicator device according to claim 1.

14. A computer program product comprising computer-readable instructions, when executed by a control device for a magnetic resonance imaging system, are configured to cause the magnetic resonance imaging device to perform the method of claim 7.

15. A computer-readable medium on which is stored program elements that, when executed by a computer unit, are configured to cause the computer unit to perform the method according to claim 7.

16. The risk indicator device according to claim 2, wherein
the risk indicator unit is configured to evaluate the risk-probability of at least one of an overdose or of contrast allergic reactions of a patient in the course of a following CT-examination, and
the risk indicator unit is configured to evaluate the risk-probability of contrast allergic reactions by using additional information of the dataset concerning at least one of (1) allergic dispositions, (2) recent lab results or (3) previous CT-examination and diagnosis of the patient.

17. The risk indicator device according to claim 2, comprising:
the data interface is configured to receive medical data types of different structures for accessing data from different sources, wherein the medical data types including data from electronic medical records, cardiovascular information systems, radiology information systems or laboratory information systems.

18. The risk indicator device according claim 2, wherein the output unit is configured to output control data for a computed tomography (CT)-system, wherein the control data includes at least one of automatic adjust contrast injection parameters or scan parameters, the control data being based on the evaluated risk-probability.

19. The risk indicator device according to claim 3, comprising:
the data interface is configured to receive medical data types of different structures for accessing data from different sources, wherein the medical data types including data from electronic medical records, cardiovascular information systems, radiology information systems or laboratory information systems.

20. The risk indicator device according claim 3, wherein the output unit is configured to output control data for a computed tomography (CT)-system, wherein the control data includes at least one of automatic adjust contrast injection parameters or scan parameters, the control data being based on the evaluated risk-probability.
